(19) United States
(12) Patent Application Publication
(10) Pub. No.: US 2020/0375681 A1
(43) Pub. Date: Dec. 3, 2020

(54) Detachable Finger-Activated Surgical Control Device

(71) Applicant: Bausch & Lomb Incorporated, Rochester, NY (US)

(72) Inventors: Ronald J. CUMMINGS-KRALIK, Wildwood, MO (US); J. Alan RITTER, Des Peres, MO (US); Michael D. HUDSPETH, Arnold, MO (US); David W. HERTWECK, Valley Park, MO (US)

(21) Appl. No.: 16/429,347

(22) Filed: Jun. 3, 2019

(51) Int. Cl. A61B 34/00

(52) U.S. Cl. CPC A61B 34/74; A61B 2017/00477; A61B 34/25

(57) ABSTRACT

A surgical control device includes a housing, a power source held within the housing, at least 1 switch mounted on a housing surface, and a transmitter held within the housing in communication with the switch. The device is for attachment to a surgical instrument, such that a user activates the switch by placing the switch and an opposing housing surface between a thumb and a finger and squeezing the thumb and finger towards each other, whereby the squeezing does not induce movement of the attached surgical instrument. The device transmits an activation signal from the switch to a remote controller for controlling a surgical function. The device also includes a removable attachment device for attaching the housing to the surgical instrument.

### FIELD

The present disclosure relates to a surgical control device removably attached to a surgical instrument with a least one finger-activated switch for transmitting an activation signal to a remote controller for controlling a surgical function.

### BACKGROUND

This section provides background information related to the present disclosure which is not necessarily prior art.

Currently, a surgeon, especially an ophthalmic surgeon, controls most surgical function and levels via a foot control that may have a variety of pedals and switches. The surgical functions and levels include ultrasonic or other surgical power, aspiration level, infusion level, illumination level, laser firing, etc. For surgeons with limited foot dexterity or that operate standing up, moving at least one surgical function and/or level control to the handpiece may be desirable.

Previous attempts have been made to place controls on surgical handpieces. Thus far, these have been buttons or slider-switches formed on or activated by pressing towards a central axis of a surgical handpiece. These previous handpiece controls create a problem for a surgeon to maintain movement control of the surgical tip while pressure is applied to the button and/or slider.

Existing handpiece actuation systems have typically required an additional power and control cable to be connected to the device and a surgical control console. The additional power and control cable creates additional strain and torque on a surgeon's hand that may lead to fatigue during surgery. The additional cable may also make handpiece manipulation more difficult and creates another potential source of tangling.

Thus, in light of the above, for certain surgeries or at particular times during a procedure it may be critical for the surgeon to know she can hold the surgical tip steady, to avoid unwanted damage to surgical tissue. Switching to a one or two-button configuration requiring a forceps-style squeezing of the button(s) balances the activation forces and allows the surgical tip to remain steady while the button(s) activate the surgical function.

Further, moving to a self-powered, wireless surgical control device allows the surgeon to have finger control of a surgical function without loss of precise handpiece tip control during activation of the surgical function. A variety of device holders will allow the surgical control device to be attached to multiple different surgical handpieces that the surgeon may use. The surgical control device may even be attached to non-powered handpieces to control surgical functions unrelated to the non-powered handpiece. Ultimately, the surgeon won't have to abandon or replace favored handpieces to take advantage of the new finger control mode.

### DRAWINGS

The drawings described are for illustrative purposes only of selected embodiments and not all possible implementations, and are not intended to limit the present disclosure.

FIG. 1 is a perspective view of an example surgical control device in accord with the present disclosure;

FIG. 2 is a partial perspective view of the surgical control device of FIG. 1 attached to a surgical handpiece;

FIG. 3 is a top view of FIG. 2 being held in a surgeon's hand;

FIG. 4 is a system view showing the surgical control device and surgical handpiece of FIG. 2 connected to an example surgical control console; and

FIG. 5 is an example circuit block diagram of a surgical control device.

Corresponding reference numerals indicate corresponding parts throughout the several views of the drawings.

### DETAILED DESCRIPTION

Example embodiments will now be described more fully referring to the accompanying drawings.

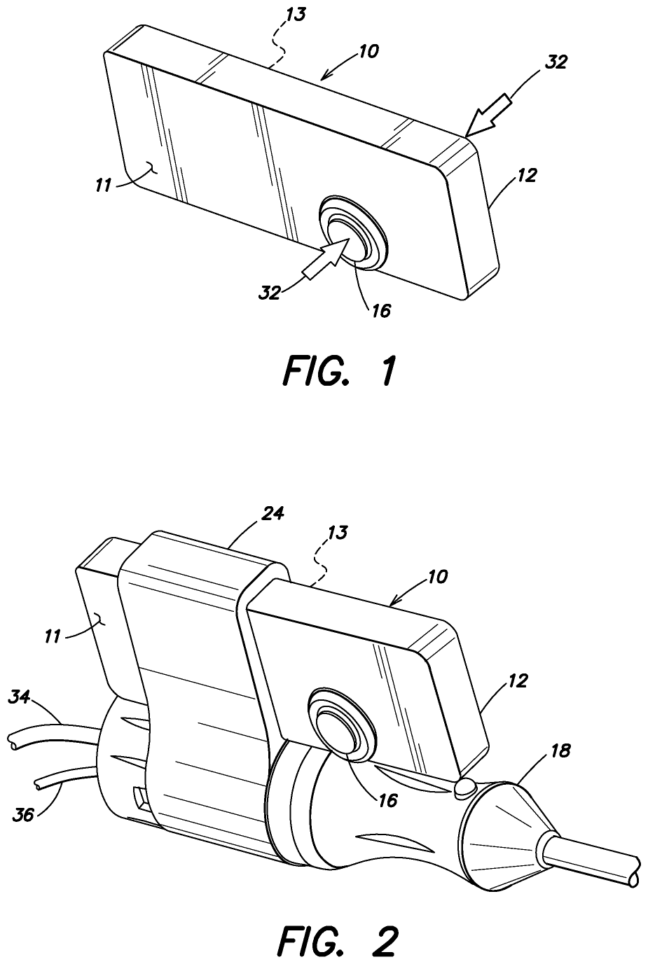

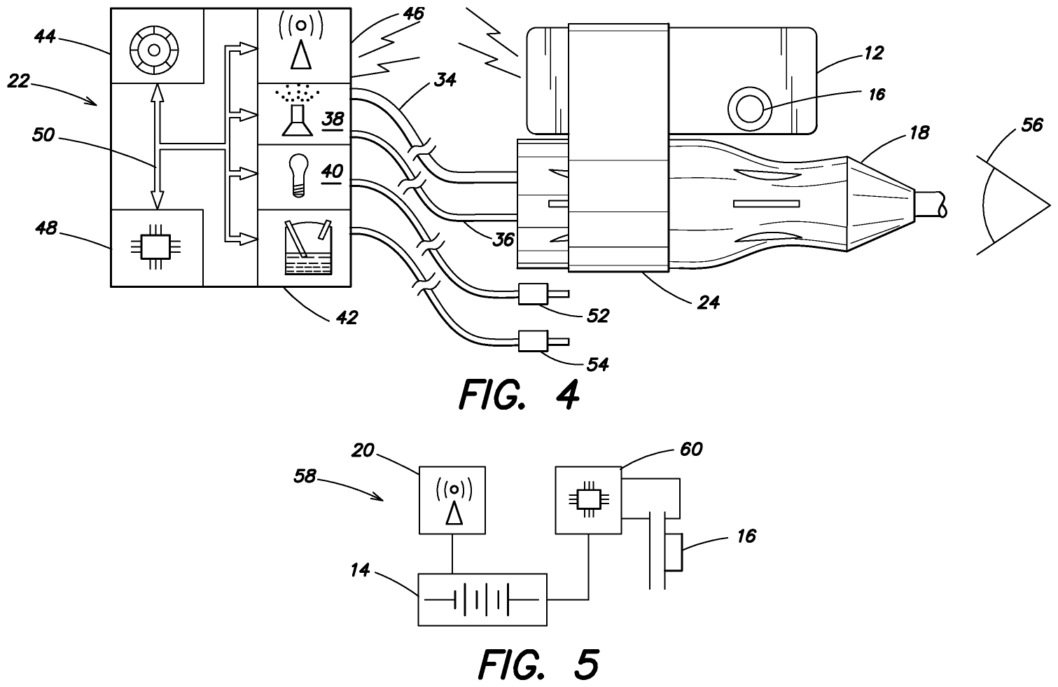

An example surgical control device 10 is shown in FIG. 1. The device 10 may include a housing 12, a power source 14 (shown in FIG. 5) held within the housing 12, at least 1 switch 16 mounted on a housing surface 11, for attachment to a surgical instrument 18 (shown in FIG. 2), a transmitter 20 (also shown in FIG. 5) held within the housing 12 in communication with the at least 1 switch 16 for transmitting an activation signal from the at least 1 switch 16 to a remote controller 22 (shown in FIG. 4) for controlling a surgical function, and a removable attachment device 24 (shown in FIG. 2) for attaching the housing 12 to the surgical instrument 18.

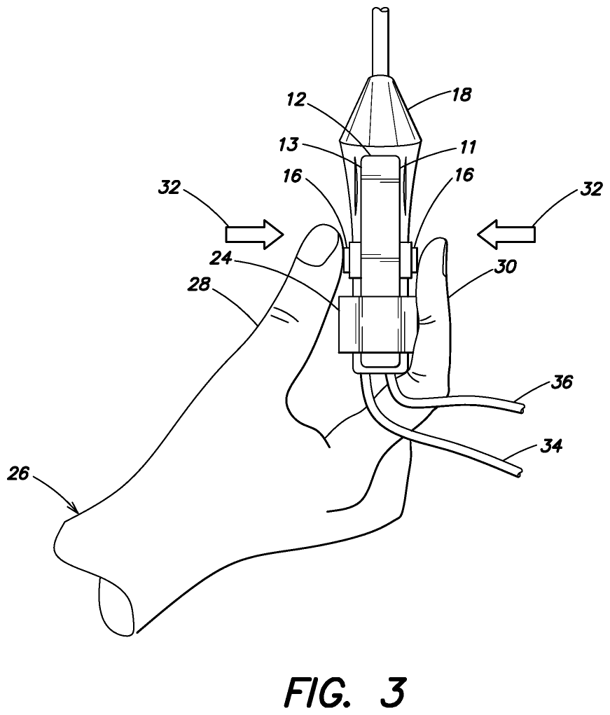

Referring to FIGS. 1-3, in accordance with the present example, a user 26 activates the switch 16 by placing the switch 16 and an opposing housing surface 13 (or a second switch 16 mounted on the opposing housing surface 13, as shown in FIG. 3) between a thumb 28 and a finger 30 and squeezing the thumb and finger towards each other, as indicated by arrows 32. Because of such a switch 16 orientation between two fingers of the user, the squeezing does not induce movement of the attached surgical instrument 18.

In use, the surgical instrument 18, may be connected to the remote controller 22 via an aspiration line 34 and an electrical power line 36, or other appropriate lines (not shown) or a surgical instrument attached to the surgical control device 10 may be completely unpowered, e.g. a forceps or scissors. The removable attachment device 24 may be a pliant band, as shown, or may be an adhesive, a snap-on attachment, a band of hook and loop material, or other appropriate structure for removably attaching device 10 to a surgical instrument.

Pressing button(s) 16 may control one or a variety of surgical functions. The particular surgical function controlled by device 10 may be programmed via the remote controller 22, shown in FIG. 4. The remote controller 22 may be a surgical console for providing and controlling a plurality of surgical functions, such aspiration and ultrasonic power 38, illumination 40, infusion/irrigation 42, gas/fluid injection (not shown), etc. The surgical function controlled may control a function of the attached surgical instrument 18 or a surgical function unrelated to surgical instrument 18, such a illumination or the infusion pressure. A user interface 44, such as a graphical user interface (GUI) may be used to program the surgical function controlled by device 10. Preferably, remote controller 22 communicates, via a wireless transmitter 46, with the transmitter 20 of device 10. Transmitter 20 and the communication with transmitter 46 may be any appropriate wireless communication scheme such as Bluetooth, infrared, Zigbee®, microwave, Wi-Fi, etc. The remote controller 22 may also include a central processing unit (CPU) 48 connected to each component or module of the remote controller 22, via a data bus 50. In operation, illumination module 40 may be connected to a fiber optic or light pipe 52 and infusion/irrigation module may be connected to a fluid supply line 54. It is noted that in some instances supply line 54 may be connected to a surgical instrument, e.g. a phacoemulsification handpiece. In any case a number of devices, e.g. surgical instrument 18, light pipe 52, and supply line 54 may be inserted into a surgical site, such as eye 56.

FIG. 5 is a partial example control circuit 58 held within surgical control device 10. Circuit 58 may include power source 14, switch(es) 16, transmitter 20 (preferably a wireless transmitter), and a microprocessor 60. Microprocessor 60 may include an integral radio, rather than the separate transmitter 20, as shown. The switches 16 may be force sensors to provide a proportional activation signal based on a range of squeezing force the thumb and finger apply to the force sensors. If switches 16 are force sensors, circuit 58 may include unshown components including MEMs based force bridge sensors and an appropriate finger contact piece, along with amplifiers and passive components to scale output voltage levels for an analog to digital (A/D) converter. The goal is to scale and limit the level of pressure needed to be supplied to the switches 16 in a repeatable, predictable way while providing a pleasant tactile feel to the user. It is believed that circuit 58 should be able to read proportional forces between 0.1 and 2.5 pound-force (0.045 and 0.45 kilogram-force). In this way circuit 58 determines a range of squeezing force applied to the force sensors.

It is noted that power source may be a battery, a capacitive quick charge circuit, a broadcast power receiver, or other appropriate power source for driving circuit 58.

The surgical control device described above provides a surgeon the ability to control a surgical function with finger control, including proportional control of the surgical function, without risking unwanted movement of the surgical instrument, especially a tip of the instrument that may be adjacent delicate tissue, e.g. the retina. The ability to maintain a steady, non-moving surgical instrument is provided by the orientation of the surgical control device's switches so that counter-balancing pressure is applied to the switch(es)/button(s) 16. This is opposed to known prior art surgical instruments with finger-activated switches that require a pressure force in a single direction, thus creating the likelihood that the surgical instrument will be moved. In addition, because the surgical control device is a separate, removably attached device, it may be attached to a variety of surgical instruments, including single-use, disposable instruments and used multiple times and control a variety of surgical functions.

The foregoing description of the embodiments has been provided for illustration and description. It is not intended to be exhaustive or to limit the disclosure. Individual elements or features of a particular embodiment are not limited to that embodiment, but, where applicable, are interchangeable and can be used in a selected embodiment, even if not specifically shown or described. The same may also be varied in many ways. Such variations are not to be deemed a departure from the disclosure, and all such modifications are included within the disclosure.

Example embodiments are provided so this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many forms and that neither should be construed to limit the disclosure. In some example embodiments, well-known processes, well-known device structures, and well-known technologies are not described in detail.

The terminology used is to describe particular example embodiments only and is not intended to be limiting. As used, the singular forms “a,” “an,” and “the” may be intended to include the plural forms, unless the context indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is described as being “on,” “engaged to,” “connected to,” or “coupled to” another element or layer, it may be directly on, engaged, connected or coupled to the other element or layer, or intervening elements or layers may be present. When an element is described as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used, the term “and/or” includes all combinations of one or more of the associated listed items.

Although the terms first, second, third, etc. may describe various elements, components, regions, layers and/or sections, these elements, components, regions, layers and/or sections should not be limited by these terms. These terms may only distinguish one element, component, region, layer or section from another region, layer or section. Terms such as “first,” “second,” and other numerical terms when used imply no sequence or order unless clearly indicated by the context. A first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used for ease of description to describe one element or feature's relationship to another element(s) or feature(s) as illustrated in the figures. Spatially relative terms may be intended to encompass different orientations of the device in use or operation besides the orientation depicted in the figures. If the device in the figures is turned over, elements described as “below” or “beneath” other elements or features would then be oriented “above” the other elements or features. The example term “below” can encompass both an orientation of above and below. The device may be otherwise oriented (rotated 90 degrees or at other orientations) and the spatially relative descriptors used, interpreted accordingly.

What is claimed is:

1. A surgical control device comprising:
a housing;
a power source held within the housing;
at least 1 switch mounted on a housing surface, for attachment to a surgical instrument, such that a user activates the switch by placing the switch and an opposing housing surface between a thumb and a finger and squeezing the thumb and finger towards each other, whereby the squeezing does not induce movement of the attached surgical instrument;
a transmitter held within the housing in communication with the at least 1 switch for transmitting an activation signal from the at least 1 switch to a remote controller for controlling a surgical function; and
a removable attachment device for attaching the housing to the surgical instrument.

2. The surgical control device of claim 1 further including a second switch mounted on the opposing housing surface.

3. The surgical control device of claim 2 wherein the switches are force sensors to provide a proportional activation signal based on a range of squeezing force the thumb and finger apply to the force sensors.

4. The surgical control device of claim 3 the housing further including a circuit for determining the range of squeezing force applied to the force sensors.

5. The surgical control device of claim 1 wherein the transmitter is a wireless transmitter.

6. The surgical control device of claim 1 wherein the remote controller is a surgical console for controlling a plurality of surgical functions.

7. The surgical control device of claim 1 wherein the controlled surgical function includes a surgical function of the attached surgical instrument.

8. The surgical control device of claim 1 wherein the removable attachment device includes one of an adhesive and a band.
